(19) United States
(12) Patent Application Publication
(10) Pub. No.: US 2003/0187471 A1
(43) Pub. Date: Oct. 2, 2003

(54) Endoscopic surgical device

(76) Inventor: Michael Cooper, New South Wales (AU)

Correspondence Address: NIXON & VANDERHYE, PC, 1100 N GLEBE ROAD, 8TH FLOOR, ARLINGTON, VA, 22201-4714, US

(21) Appl. No.: 10/344,844

(22) PCT Filed: Aug. 13, 2001
(86) PCT No.: PCT/AU01/00988

(30) Foreign Application Priority Data
Aug. 14, 2000 (AU) PQ 9410

(51) Int. Cl. A61B 17/34

(52) U.S. Cl. 606/185

(57) ABSTRACT

An endoscopic surgical instrument (10) for insertion into a body of a patient, the endoscopic surgical instrument (10) including a capture means for capturing matter characteristic of damage to a structure of the body caused during an endoscopic procedure, such as puncture of an artery or a bowel. The instrument may be a trocar (15) with a plunger (33) that captures matter via apertures (32), the matter being analysed for the presence of matter characteristic of damage, such as bacteria or faecal matter in the case of injury to the bowel. Other embodiments include absorbent swabs (19, 53) or holes (51, 52) on the distal end of a trocar (15), or an internal rim (23) on the distal end of a cannula (11).

### FIELD OF THE INVENTION

The present invention relates to a device and method for facilitating the detection of damage to a structure of the body during an endoscopic surgical procedure.

### BACKGROUND ART

Minimally invasive (endoscopic) surgery usually involves the use of a surgical assembly comprising a trocar positioned within the lumen of a cannula. Typically, the trocar is a rod-like solid member which has a sharpened or pointed end for assisting in puncturing the wall of a body cavity. Upon puncture of the wall, the trocar and cannula may be advanced through the wall and into a body cavity thereby creating a small opening in the wall of the body cavity. When the assembly is correctly positioned, the trocar is withdrawn from the cannula and the lumen of the cannula used as an entrance for endoscopic surgical instruments. In other cases, a short cut down using a scalpel is made before a cannula containing a blunt trocar is advanced through the cavity wall and into the body cavity.

Prior to use of the trocar, the body cavity is often insufflated with an inert gas such as carbon dioxide (CO₂) or filled with a fluid such as normal saline or glycine, to facilitate access to the organs within the body cavity. Upon completion of the surgical procedure, the cannula is removed from the opening and the body cavity desufflated or drained as the case may be.

In many cases, the above minimally invasive procedure is favoured over “open” or conventional surgery due to a lower post-operative morbidity, shorter post-operative stay, less pain, decreased cost and a quicker recovery period.

While often favoured for the above reasons, there are still many risks associated with minimally invasive surgery including the risk of damage to a structure of the body (artery, bowel etc..).

In addition to damage caused by trocars, there are many cases of damage by insufflation needles and other laparoscopic instruments. Typically, damage by a trocar will occur when the trocar or trocar/cannula assembly is first advanced through the wall of the body cavity. Particularly, the advancing pointed end of the trocar may catch or puncture a structure lying beneath the cavity wall. Where a scalpel is used to form a cut down, there is also a risk, for example, that the scalpel will damage a structure of the body, such as a portion of underlying bowel.

Establishing a pneumoperitoneum by inserting an insufflation needle and introducing a fluid also has many risks. In one study ¹ data was collected on 14,243 patients undergoing various standard laparoscopic procedures between 1995 and 1997. This database was investigated with special regard to intraabdominal complications caused by trocars and insufflation needles. The results of the study showed 22 trocar and 4 needle injuries. ¹The Swiss Association for Laparoscopic and Thoracoscopic Surgery (SALTS) Aarberg Hospital, Aarberg, Switzerland 1995 to 1997.

Further studies suggest that only around half of these injuries are recognised intraoperatively. If not recognised intraoperatively and repaired immediately, such damage increases morbidity and may lead to the death of the patient.

In an attempt to prevent injury of the bowel, devices including a spring back sheath to cover the pointed end of the trocar after insertion into the body cavity have been developed. However, such devices cannot shield the trocar while it is puncturing the wall of a body cavity. Accordingly, if the bowel moves or if a portion of the bowel adheres to the wall of the cavity there is still a risk of injury as the trocar is advanced into the body cavity.

The present invention addresses the problems of the prior art and provides a means to detect damage to a structure of the body during an endoscopic surgical procedure rather than attempt to prevent such damage.

### DISCLOSURE OF THE INVENTION

In a first aspect, the present invention is an endoscopic surgical instrument for insertion into a body of a patient, the endoscopic surgical instrument including a proximal end, a distal end and at least one capture means for capturing matter characteristic of damage to a structure of the body caused during an endoscopic procedure.

In one embodiment, the instrument captures matter characteristic of damage caused on or during insertion of the endoscopic surgical instrument.

In another embodiment, the endoscopic surgical instrument is adapted to capture matter characteristic of damage caused during the endoscopic procedure wherein the damage is caused by an instrument other than the endoscopic surgical instrument of the invention. In a preferred embodiment, the instrument is adapted to capture matter characteristic of damage caused on or during insertion of the endoscopic surgical instrument in addition to damage caused by an instrument other than the endoscopic surgical instrument used in the procedure.

The endoscopic surgical instrument of the invention may be a trocar. In this embodiment, damage to a body structure may be caused by the insertion of the trocar into the body cavity. Typically a distal end of any surgical trocar may be pointed to pierce the skin and tissue of a patient thereby enabling insertion of the trocar into a body cavity of said patient. It is envisaged that the damage to a body structure, such as the bowel, may be caused by the pointed distal end of the trocar.

Preferably, the at least one capture means comprises a chamber located internal the trocar. In this embodiment, the chamber is in fluid communication with the surrounding environment by way of at least one aperture located on a wall of the trocar. The chamber may extend the entire length of the trocar or only a portion thereof. Preferably, the chamber extends from the distal end of the trocar wherein the at least one aperture is located at the distal end of the trocar. In use, when the trocar is advanced through the tissue of a patient, it is envisaged that some of the matter surrounding the trocar will enter the chamber through the at least one aperture. Such matter may include indicators of damage to a body structure e.g faecal matter ill the case of injury to the bowel.

In a further embodiment, the chamber receives a plunger member. The plunger member may be moveable along a length of the chamber either in a first direction towards the distal end of the trocar and in a second direction away from the distal end of the trocar. The plunger member may be caused to move by several means including actuation of a push-pull rod which is connected to the plunger, the push-pull rod being located outside the body of the patient to facilitate manipulation of the rod by a user.

When the trocar is removed from the body of a patient, the plunger member is preferably moved in the first direction towards the distal end of the trocar. The force of the plunger against the contents of the chamber forces the contents through the at least one aperture in the wall of the trocar. In this embodiment, any matter which enters the chamber of the trocar during the procedure may be subsequently expelled from the chamber following the procedure.

In a further embodiment, the plunger member may be moved in the first direction towards the distal end of the trocar either immediately prior to use or during use of the trocar. In this embodiment, the plunger member is held in a first position adjacent the distal end of the trocar until the user requires a sample of body matter surrounding the trocar. The plunger may then be moved in the second direction thereby drawing matter from the region surrounding the trocar through the at least one aperture and into the chamber. Accordingly, rather than relying upon matter to enter the chamber during the procedure, this embodiment provides a means of actively obtaining a sample of the surrounding matter.

The wall of the chamber may further include al outlet. Typically, the outlet is positioned in a portion of the wall of the chamber which is adjacent the proximal end of the trocar. In this embodiment, when the plunger member is in the first position, the outlet is not in communication with the contents of the chamber. When the plunger member is moved in the second direction to a second position. the aperture is brought into communication with the contents of the chamber and the contents of the chamber caused to empty from the chamber through the outlet. The outlet may extend to or be in fluid communication with a trap means which collects the contents of the chamber for analysis. This particular embodiment may be particularly useful in the collection of gaseous or aerated material from the body of a patient.

In a further embodiment of the invention, the trocar includes a tip at its distal end wherein the tip is made from a perspex material or any suitable material which allows a user to view the area surrounding the distal end of the trocar through the tip. The at least one capture means of this embodiment would preferably comprise a ridge or hole or a series of ridges or holes located on the external surface of the tip of the trocar.

The at least one capture means may further comprise a cotton bud or swab located adjacent the distal end of the trocar. Surrounding matter from the body cavity may adhere to the cotton bud or swab when the trocar is inserted into the body cavity. Upon removal of the trocar from the body cavity, the matter from the bud or swab may be collected and analysed to determine the presence of matter characteristic of damage. It is envisaged, however, that the at least one capture means could take other forms and may be positioned at other sites on the trocar.

In a further embodiment, the endoscopic surgical instrument is a cannula having a channel extending from a proximal end to a distal end. In one embodiment, damage to a body structure may be caused by the insertion of the cannula into the body cavity of a patient. In another embodiment, the cannula is adapted to capture matter characteristic of damage caused by other instruments inserted through the channel of the cannula into the body cavity, such as scissors, clamps and the like.

Preferably, the at least one capture means is positioned at the distal end of the cannula and may comprise a number of structures. For example, the at least one capture means may include a portion of a wall of the distal end of the cannula which extends into the channel to form a rim around the distal end. Upon removal of the cannula from the cavity, the matter may be removed and analysed.

In a further embodiment, the at least one capture member comprises an absorbent material connected to a portion of tie wall of the distal end of the cannula.

In a further embodiment of the invention, the at least one capture means may be located adjacent the proximal end of the cannula. In this embodiment the capture means may be positioned outside the body cavity throughout the endoscopic procedure. The capture means of this embodiment is preferably adapted to capture gaseous or aerated matter, said matter being expelled under pressure from the body during the endoscopic procedure.

In a second aspect, the present invention provides an insufflation device for use in endoscopic surgery, said insufflation device including a proximal end, a distal end and at least one capture means for capturing matter characteristic of damage to a structure of the body caused during an endoscopic procedure.

Insufflation is a preliminary step in an endoscopic procedure and particularly endoscopic surgery of the abdomen. The process of insufflation “retracts” the anterior abdominal wall thereby exposing the operative field. The insufflation device typically delivers carbon dioxide (CO₂) as this gas is rapidly cleared from the body by the lungs and will not support combustion. The step of insufflation provides an opportunity to test for any damage to a structure of the body caused during entry of the trocar, cannula or insufflation device.

The insufflation device preferably includes an internal lumen extending from an opening in the distal end towards the proximal end with an inner member spring loaded within the internal lumen of the insufflation device. In this embodiment, the at least one capture means comprises at least a portion of the inner member.

In a further embodiment, the at least one capture means comprises an absorbent member positioned on the inner member. Alternatively, the at least one capture means may comprise a chamber within the inner member.

As the inner member is spring loaded within the internal lumen of the insufflation member it may be moved from a first capture position wherein the distal end of the inner member extends beyond the distal end of the insufflation device to a second position wherein the distal end of the inner member is drawn through the opening and into the internal lumen of the insufflation device. This enables a user to control the timing of the capture of surrounding matter.

In a third aspect, the present invention provides an endoscopic surgical assembly for insertion into a body of a patient, the assembly including:

a cannula having a channel extending from a proximal end to a distal end, the cannula further including at least one capture means for capturing matter characteristic of damage to a body structure caused on or during insertion of the assembly; and

a trocar mounted inside the channel of the cannula and having a proximal end, a distal end and at least one capture means for capturing matter characteristic of damage to a body structure caused on or during insertion of the assembly.

In an embodiment of the third aspect of the invention, the damage to the structure of the body may be caused by either the trocar or the cannula or both the trocar and cannula or by any other instrument used during an endoscopic procedure.

A distal end of any surgical trocar is typically pointed to pierce the skin and tissue of a patient thereby enabling insertion of the trocar into a body cavity of said patient. It is envisaged that the damage to a body structure, such as the bowel, may be caused by the pointed distal end of the trocar.

Preferably, the at least one capture means of the trocar comprises a chamber located internal the trocar. In this embodiment, the chamber is in fluid communication with the surrounding environment by way of at least one aperture located on a wall of the trocar. The chamber may extend the entire length of the trocar or only a portion thereof. Preferably, the chamber extends from the distal end of the trocar wherein the at least one aperture is located at the distal end of the trocar. In use, when the trocar is advanced through the tissue of a patient, it is envisaged that some of the matter surrounding the trocar will enter the chamber through the at least one aperture. Such matter may include indicators of damage to a body structure e.g faecal matter in the case of injury to the bowel.

In a further embodiment, the chamber receives a plunger member. The plunger member may be moveable along a length of the chamber either in a first direction towards the distal end of the trocar and in a second direction away from the distal end of the trocar. The plunger member may be caused to move by several means including actuation of a push-pull rod which is connected to the plunger, the push-pull rod being located outside the body of the patient to facilitate manipulation of the rod by a user.

When the trocar is removed from the body of a patient, the plunger member is preferably moved in the first direction towards the distal end of the trocar. The force of the plunger against the contents of the chamber forces the contents through the at least one aperture in the wall of the trocar. In this embodiment, any matter which enters the chamber of the trocar during the procedure may be subsequently expelled from the chamber following the procedure.

In a further embodiment, the plunger member may be moved in the first direction towards the distal end of the trocar either immediately prior to use or during use of the trocar. In this embodiment, the plunger member is held in a first position adjacent the distal end of the trocar until the user requires a sample of body matter surrounding the trocar. The plunger may then be moved in the second direction thereby drawing matter from the region surrounding the trocar through the at least one aperture and into the chamber. Accordingly, rather than relying upon matter to enter the chamber during the procedure, this embodiment provides a means of actively obtaining a sample of the surrounding matter

The wall of the chamber may further include an outlet. Typically, the outlet is positioned in a portion of the wall of the chamber which is adjacent the proximal end of the trocar. In this embodiment, when the plunger member is in the first position, the outlet is not in communication with the contents of the chamber. When the plunger member is moved in the second direction to a second position, the aperture is brought into communication with the contents of the chamber and the contents of the chamber caused to empty from the chamber through the outlet. The outlet may extend to or be in fluid communication with a trap means which collects the contents of the chamber for analysis. This particular embodiment may be particularly useful in the collection of gaseous or aerated material from the body of a patient.

In a further embodiment of the invention, the trocar includes a tip at its distal end wherein the tip is made from a perspex material or any suitable material which allows a user to view the area surrounding the distal end of the trocar through the tip. The at least one capture means of this embodiment would preferably comprise a ridge or hole or a series of ridges or holes located on the external surface of the tip of the trocar.

The at least one capture means of the trocar may further comprise a cotton bud or swab located adjacent the distal end of the trocar. Surrounding matter from the body cavity may adhere to the cotton bud or swab when the trocar is inserted into the body cavity. Upon removal of the trocar from the body cavity, the matter from the bud or swab may be collected and analysed to determine the presence of matter characteristic of damage. It is envisaged, however, that the at least one capture means could take other forms and may be positioned at other sites on the trocar.

In a further embodiment, at least one capture means of the cannula is positioned at the distal end of the cannula and may comprise a number of structures. For example, the at least one capture means may include a portion of a wall of the distal end of the cannula which extends into the channel to form a rim around the distal end. Upon removal of the cannula from the cavity, the matter may be removed and analysed.

In a further embodiment, the at least one capture member comprises an absorbent material connected to a portion of the wall of the distal end of the cannula.

In a further embodiment of the invention, the at least one capture means may be located adjacent the proximal end of the cannula. In this embodiment the capture means may be positioned outside the body cavity throughout the endoscopic procedure. The capture means of this embodiment is preferably adapted to capture gaseous or aerated matter, said matter being expelled under pressure from the body during the endoscopic procedure.

In a fourth aspect, the present invention is an endoscopic surgical procedure, the procedure including the steps of:

(i) inserting the endoscopic surgical instrument of the first aspect into a body cavity;

(ii) sampling matter present within the body cavity; and

(iii) analysing the matter to determine if it is characteristic of damage to a structure within the body cavity.

In one embodiment of the fourth aspect, the sampling step is continuously or periodically performed throughout the endoscopic procedure.

In a further embodiment, the step of analysing the matter is continuously or periodically performed during the endoscopic surgical procedure.

In another embodiment, the procedure further involves sampling the gaseous contents of the body cavity and analysing the gas samples.

In a still further embodiment, the step of sampling and/or the step of analysing matter is performed immediately after insertion of the surgical instrument into the body cavity.

In yet a further embodiment, the step of sampling and/or the step of analysing matter is performed immediately prior to withdrawal of the surgical instrument at the end of the procedure.

In a fifth aspect, the present invention is a method for detecting damage to a structure of a body of a patient during an endoscopic procedure including the steps of:

(i) advancing the surgical assembly of the third aspect of the invention through a body cavity wall of a patient so as to create an entry port;

(ii) correctly positioning the assembly for surgical access to a target site and subsequently removing the trocar from the channel of the cannula; and

(iii) analysing the contents of the capture means of the trocar to establish whether damage has occurred to a structure of the body.

In a further embodiment, the method includes the further steps of:

(iv) inserting the. insufflation device of the second aspect through the cannula and insufflating the body cavity with a suitable gas or liquid:

(v) withdrawing the insufflation device and analysing the contents of the at least one capture means of the insufflation device to establish whether damage has occurred to a structure of the body;

(vi) withdrawing the cannula at the end of the endoscopic surgical procedure and analysing the contents of the at least one capture means of the cannula.

In an embodiment of each aspect of the invention, the endoscopic surgical instrument, the endoscopic surgical assembly or the insufflation device may be used in a wide range of procedures including but not limited to laparoscopy, arthroscopy, thoracoscopy and hysteroscopy.

The at least one capture means of each aspect of the present invention, in addition to capturing matter, may further include a means to analyse said matter. For example, in one form, the capture means may include a biosensor or matter-sensitive material which upon contact with specific matter will change property, such as colour. Upon sighting the property change a user would recognise that damage to a structure of the body had occurred.

Typically, the matter indicative of damage to a structure of the body will vary with each surgical procedure. For example, injury of the bowel will cause the release of different matter to that released when, for example, the bladder is inadvertently injured.

During laparoscopic surgery, the bowel may be injured leading to the release of a number of bacteria such as Escherichia coli. Capture and identification of such bacteria will indicate to the user that the bowel has likely been damaged. during the procedure. Preferably, an indication of the concentration of the bacteria is also provided to enable a surgeon to make a proper assessment of the situation. For example, if only a low concentration of bacteria are detected, there is a possibility that such bacteria were picked up from the skin or other sources during the procedure rather than resulting from injury of the bowel.

### BRIEF DESCRIPTION OF THE DRAWINGS

FIG. 1 is a perspective view of a typical device used in an endoscopic procedure.

FIG. 2 is a side elevational view of one embodiment of the present invention.

FIG. 3 is a side elevational view of part of the embodiment depicted in FIG. 2.

FIG. 4 is a cross-sectional view of a further embodiment of the present invention.

FIG. 5 is a side elevational view of a still further embodiment of the present invention.

FIG. 6 is a cross-sectional top plan view through I-I of FIG. 5.

FIG. 7 is a cross-sectional side view of a further embodiment of the invention.

FIGS. 8a and 8b are cross-sectional side views of another embodiment of the invention.

FIG. 9 and 10 are cross-sectional side views through a further aspect of the present invention.

### DESCRIPTION OF THE INVENTION

One example of an endoscopic surgical device according to the present invention is depicted as 10 in the drawings. The device 10 comprises a cannula 11 having a central channel 12 extending from a proximal end 13 to a distal end 14 and, further, a trocar 15 mounted inside the central channel 12 of the cannula 11. The trocar 15 is preferably a rod-like solid member having a sharpened or pointed distal end 16 which extends beyond the distal end 14 of the cannula 11 and acts to puncture a wall of a body cavity 17 at commencement of an endoscopic procedure. While not depicted, it will be appreciated by a person skilled in the art that entry to the body cavity may also be achieved by forming a cut down using a scalpel at an appropriate location and then inserting a cannula, such as cannula 11, or a cannula/blunt trocar combination into the cavity.

During the endoscopic procedure, the depicted cannula 11 and trocar 15 are advanced through the body cavity wall 17 and into the body cavity 20. The trocar 15 may then be withdrawn from the central channel 12 of the cannula 11. With the trocar 15 removed, the central channel 12 of the cannula 11 can act as a port for the introduction of endoscopic surgical instruments. Before the surgery commences, however, the body cavity 20 is normally, depending on its nature, insufflated with an inert gas such as carbon dioxide (CO₂) or filled with a fluid such as normal saline or glycine, to facilitate surgical access to the structures of the body cavity 20. Upon competition of the surgery, the cannula 11 is removed from the opening in the cavity wall and the body cavity 20 desufflated or drained as the case may be.

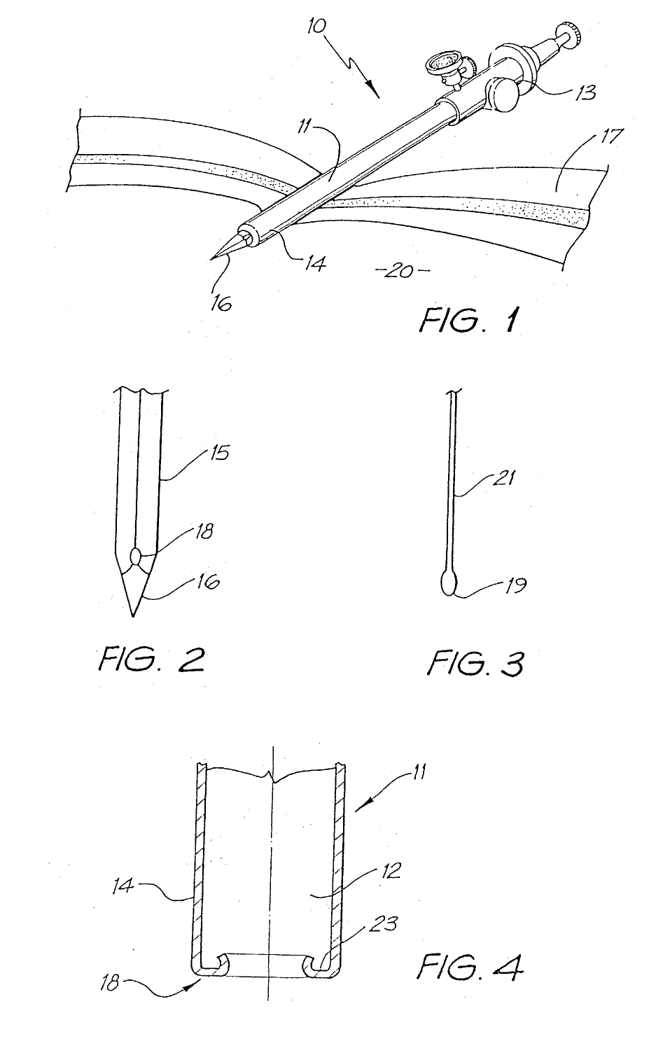

In an embodiment of the present invention, the trocar 15 includes a capture member 18 preferably located adjacent distal end 16 of trocar 15. In the embodiment depicted in FIG. 3, the capture member 18 is a bud or swab 19 held at the distal end 16 by an elongate member 21. When the trocar is inserted through the cavity wall 17 and into the body cavity 20, matter from the body cavity 20 will adhere to the capture member 18. When the trocar 15 is removed from the central channel 12 of the cannula 11, the capture member 18 may be removed and the contents of the bud or swab 19 analysed as discussed below.

In an alternate embodiment depicted in FIG. 4, the cannula 11 also comprises a capture member 18 preferably located adjacent its distal end 14. Particularly, the capture member 18 comprises a rim 23 around distal end 14. In this embodiment, at least some matter from the body cavity 20 will likely collect in the rim 23 and can be analysed at the end of the endoscopic procedure when the cannula 11 is removed.

It is also envisaged that the cannula 11 may also comprise a capture member 18 located adjacent proximal end 13. In this embodiment, the capture member 18 comprises a biosensor or dish or other like structure having within it a collection member 25 adapted to sample matter from a gaseous stream. The collection member 25 collects certain matter of the body cavity which- is released through the opening in the cavity wall in a pressurised, gaseous form.

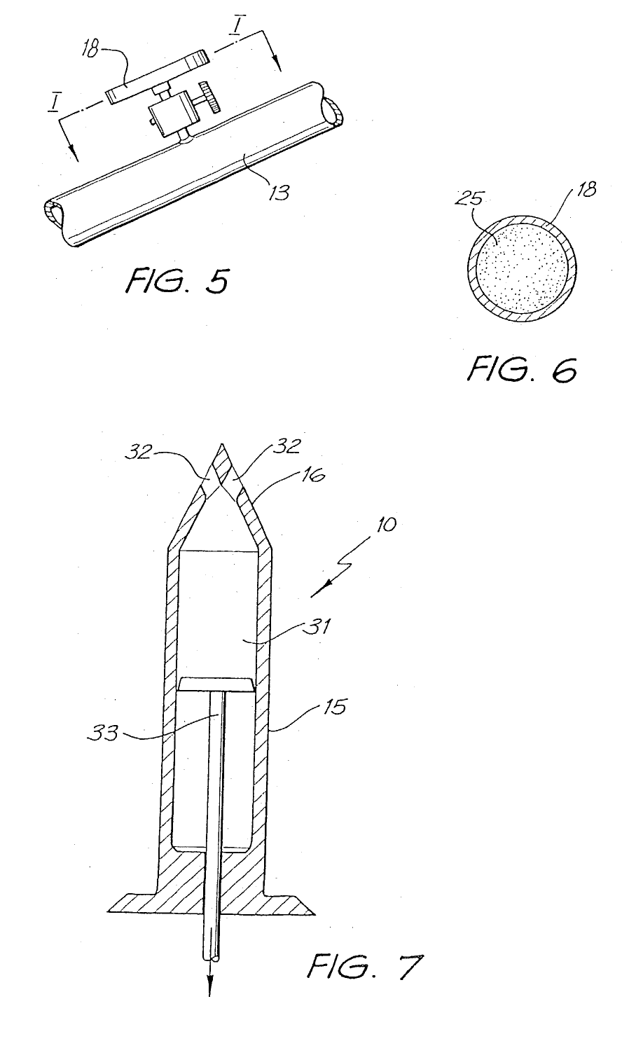

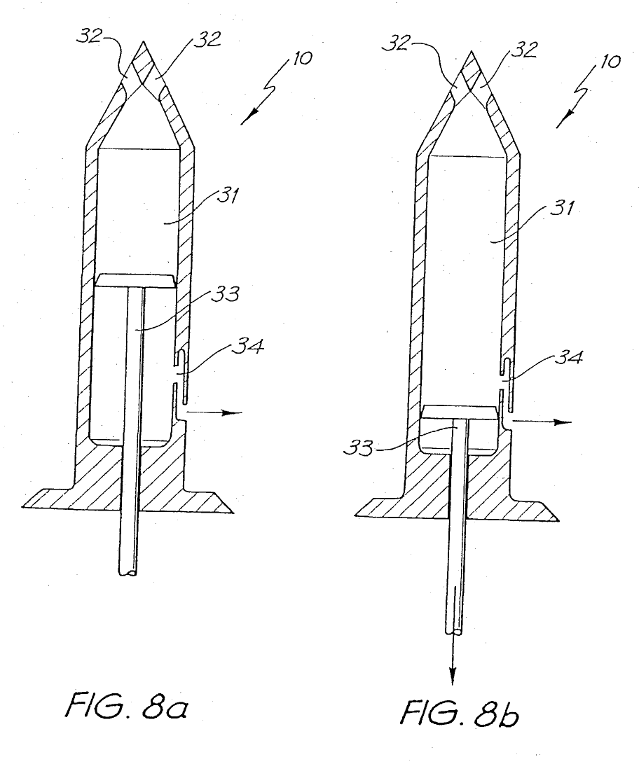

In a further embodiment of the invention depicted in FIG. 7, the capture member 18 comprises a chamber 31 located internal the trocar 15. In this embodiment, the chamber 31 is in fluid communication with the environment external the trocar 15 by way of apertures 32. The chamber 31 extends from the apertures 32 at the distal end 16 of the trocar 15. In use, when the trocar is advanced through the tissue of a patient, it is envisaged that some of the matter surrounding the trocar 15 will enter the chamber. Such matter will likely include indicators of damage to a body structure, e.g faecal matter in the case of injury to the bowel.

The chamber 31 receives a plunger member 33. The plunger member 33 may be caused to move along a length of the chamber 31 upon activation, An example of such activation includes movement of the plunger member 33 by a push-pull rod (not shown) which may be manipulated by a user.

When the trocar 15 is removed from the body of a patient, the user may push the rod thereby causing the plunger member 33 to move in a direction towards the distal end 16 of the trocar 15. The force of the plunger member 33 against the contents of the chamber forces the contents through apertures 32.

In a further embodiment depicted in FIGS. 8a and 8b, the plunger member 33 is first moved in a direction towards the distal end 16 of the trocar 15 either immediately prior to use or during use of the trocar. In this embodiment, the plunger member 33 is held in the first position as depicted in FIG. 8a until the user requires sampling of body matter around the trocar 15. The plunger member 33 may then be drawn back towards the proximal end of the trocar thereby drawing matter from the region surrounding the trocar 15 through apertures 32 and into the chamber 31.

The wall of the chamber 31 further includes an outlet 34. When the plunger member 33 is in the first position, the outlet 34 is not in communication with the contents of the chamber 31 (see FIG. 8a). When the plunger member 33 is drawn back towards the proximal end of tie trocar 15 as depicted in FIG. 8b, the outlet 34 is brought into communication with the contents of the chamber 31 and the contents of the chamber 31 caused to empty from the chamber through the outlet 34. The outlet may extend to or be in fluid communication with a trap means (not shown) which collects the contents of the chamber for analysis. This particular embodiment may be particularly useful in the collection of gaseous or aerated material from the body of a patient.

The present invention also provides an insufflation device for use in endoscopic surgery as generally depicted as 41 in FIG. 9. The insufflation device 41 has a proximal end 42, a distal end 43 and a capture member 18 for capturing matter characteristic of damage to a structure of the body caused during an endoscopic procedure.

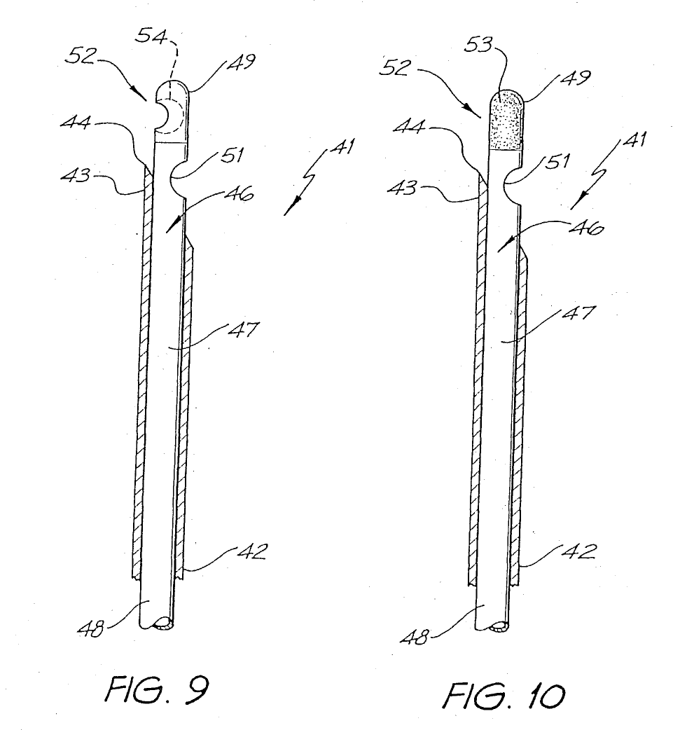

The insufflation device has a sharp tip 44 at a distal end 43 of the insufflation device 41. The insufflation device 41 further includes an internal lumen (not shown) extending from an opening 46 in the distal end 43 towards the proximal end 42 of the insufflation device 41. An inner member 47 is spring loaded within the internal lumen of the insufflation device 41. The capture member 18 comprises at least a portion of inner member 47. More specifically, the inner member 47 is a hollow structure having a proximal end 48 and a distal end 49 with an aperture 51 in a portion of the distal end 49. Gas such as carbon dioxide or a liquid such as saline may be pumped through the aperture 51 and into the body cavity of a patient. As discussed above, the process of insufflation provides the surgeon with a good operative view of the site. While the inner member 47 facilitates the transfer of fluid in this manner, surrounding matter and in particular, surrounding gases may enter the aperture 51 of the inner member 47. Such matter may then be tested to determine whether it is an indicator of damage to a structure of the body. Alternatively, as depicted in FIGS. 9 and 10, the capture member may comprise a separate collection member 52 which may be an absorbent pad or swab 53 or a chamber 54.

As the inner member 47 is spring loaded within the internal lumen 45, it may be moved from a first capture position wherein the distal end 49 of the inner member extends beyond the distal end 43 of the insufflation device 41 to a second position wherein the distal end 49 of the inner member is drawn through the opening 46 of the insufflation device 41 and into the internal lumen 45 of the insufflation device 41. This enables a user to control the timing of the capture of surrounding matter.

Clearly, each capture member 18 will collect a range of matter including the expected skin cells, blood cells etc.. The aim of the present invention, however, is to detect injury of a structure of body cavity 20 during an endoscopic procedure. Accordingly, the contents of each capture member 18 should be analysed for any matter characteristic of such injury. During laparoscopic procedures. for example, the presence of certain bacteria or bowel contents would alert the surgeon to the possibility of injury to the bowel. In such instances, the surgeon could assess the situation and repair any damage caused by the procedure.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. An endoscopic surgical instrument for insertion into a body of a patient, the endoscopic surgical instrument including a proximal end, a distal end and at least one capture means for capturing matter characteristic of damage to a structure of the body caused during an endoscopic procedure.

2. The endoscopic surgical instrument of claim 1 wherein the instrument captures matter characteristic of damage caused on or during insertion of the endoscopic surgical instrument into the body of the patient.

3. The endoscopic surgical instrument of claim 1 or claim 2 wherein the endoscopic surgical instrument is a trocar.

4. The endoscopic surgical instrument of claim 3 wherein the at least one capture means comprises a chamber located internal the trocar.

5. The endoscopic surgical instrument of claim 4 wherein the chamber is in fluid communication with the surrounding environment through at least one aperture positioned on a wall of the trocar.

6. The endoscopic surgical instrument of claim 5 wherein the chamber extends from the distal end of the trocar and wherein the at least one aperture is located at the distal end of the trocar.

7. The endoscopic surgical instrument of any one of claims 4 to 6 wherein the chamber receives a plunger member.

8. The endoscopic surgical instrument of claim 7 wherein the plunger member is moveable along a length of the chamber either in a first direction towards the distal end of the trocar or a second direction away from the distal end of the trocar.

9. The endoscopic surgical instrument of claim 8 wherein matter from the body which has entered the chamber through the at least one aperture during the endoscopic procedure is expelled from the chamber through the at least one aperture following withdrawal of the trocar from the body, by movement of the plunger in the first direction.

10. The endoscopic surgical instrument of claim 8 wherein matter from the area surrounding the trocar is drawn into the chamber through the at least one aperture by movement of the plunger in the second direction from a first position to a second position.

11. The endoscopic surgical instrument-of claim 10 wherein a wall of the chamber has an outlet.

12. The endoscopic surgical instrument of claim 11 wherein when the plunger member is in the first position, the outlet is not in communication with the contents of the chamber and when the plunger member is moved in the second direction to the second position, the outlet is brought into communication with the contents of the chamber and the contents of the chamber caused to empty from the chamber through the outlet.

13. The endoscopic surgical instrument of claim 12 wherein the outlet extends to or is in fluid communication with a trap means which collects the contents of the chamber for subsequent analysis.

14. The endoscopic surgical instrument of any one of claims 3 to 13 wherein the trocar has a tip at its distal end, the tip being made from a perspex material or any other material which enables a user to view the area surrounding the distal end of the trocar through the tip.

15. The endoscopic surgical instrument of claim 14 wherein the at least one capture means comprises a ridge or hole or a series of ridges or holes located on an external surface of the tip of the trocar.

16. The endoscopic surgical instrument of claim 3 wherein the at least one capture means comprises a cotton bud or swab located adjacent the distal end of the trocar.

17. The endoscopic surgical instrument of claim 1 or claim 2 wherein the instrument is a cannula having a channel extending from a proximal end to a distal end.

18. The endoscopic surgical instrument of claim 17 wherein the at least one capture means is positioned at the distal end of the cannula.

19. The endoscopic surgical instrument of claim 17 or claim 18 wherein the at least one capture means includes a portion of a wall of the distal end of the cannula which extends into the channel to form a rim around the distal end of the cannula.

20. The endoscopic surgical instrument of claim 17 or claim 18 wherein the at least one capture member comprises an absorbent material connected to a portion of a wall of the distal end of the cannula.

21. The endoscopic surgical instrument of claim 17 wherein the at least one capture means is positioned at a proximal end of the cannula.

22. An insufflation device for use in endoscopic surgery, said insufflation device including a proximal end, a distal end and at least one capture means for capturing matter characteristic of damage to a structure of the body caused during an endoscopic procedure.

23. The insufflation device of claim 22 further having an internal lumen which extends from an opening in the distal end to the proximal end.

24. The insufflation device of claim 23 further comprising all inner member which is spring loaded within the internal lumen of the insufflation device.

25. The insufflation device of claim 24 wherein the at least one capture means comprises at least a portion of the inner member.

26. The insufflation device of claim 25 wherein the at least one capture means comprises an absorbent member positioned on the inner member.

27. The insufflation device of claim 25 wherein the at least one capture means comprises a hollow chamber within the inner member.

28. An endoscopic surgical assembly for insertion into a body of a patient, the assembly including:
a cannula having a channel extending from a proximal end to a distal end, the cannula further including at least one capture means for capturing matter characteristic of damage to a body structure caused on or during insertion of the assembly; and
a trocar mounted inside the channel of the cannula and having a proximal end, a distal end and at least one capture means for capturing matter characteristic of damage to a body structure caused on or during insertion of the assembly.

29. The endoscopic surgical assembly of claim 28 wherein the at least one capture means of the trocar comprises a chamber located internal the trocar.

30. The endoscopic surgical assembly of claim 28 wherein the chamber is in fluid communication with the surrounding environment trough at least one aperture located on a wall of the trocar.

31. The endoscopic surgical assembly of claim 30 wherein the chamber extends from the distal end of the trocar and wherein the at least one aperture is located at the distal end of the trocar.

32. The endoscopic surgical assembly of any one of claims 29 to 31 wherein the chamber receives a plunger member.

33. The endoscopic surgical assembly of claim 32 wherein the plunger member is moveable along a length of the chamber either in a first direction towards the distal end of the trocar or a second direction away from the distal end of the trocar.

34. The endoscopic surgical assembly of claim 33 wherein matter from the body which has entered the chamber through the at least one aperture during the endoscopic procedure is expelled from the chamber through the at least one aperture following withdrawal of the trocar from the body, by movement of the plunger in the first direction.

35. The endoscopic surgical assembly of claim 33 wherein matter from the area surrounding the trocar is drawn into the chamber through the at least one aperture by movement of the plunger in the second direction from a first position to a second position.

36. The endoscopic surgical assembly of claim 35 wherein a wall of the chamber has an outlet.

37. The endoscopic surgical assembly of claim 36 wherein when the plunger member is in the first position, the outlet is not in communication with the contents of the chamber and when the plunger member is moved in the second direction to the second position, the outlet is brought into communication with the contents of the chamber and the contents of the chamber caused to empty from the chamber through the outlet.

38. The endoscopic surgical assembly of claim 37 wherein the outlet extends to or is in fluid communication with a trap means which collects the contents of the chamber for subsequent analysis.

39. The endoscopic surgical assembly of any one of claims 28 to 38 wherein the trocar has a tip at its distal end, the tip being made from a perspex material or any other material which enables a user to view the area surrounding the distal end of the trocar through the tip.

40. The endoscopic surgical assembly of claim 39 wherein the at least one capture means comprises a ridge or hole or a series of ridges or holes located on an external surface of the tip of the trocar.

41. The endoscopic surgical assembly of claim 28 wherein the at least one capture means of the trocar comprises a cotton bud or swab positioned at the distal end of the trocar.

42. The endoscopic surgical assembly of claim 28 wherein the at least one capture means of the cannula is positioned at the distal end of the cannula.

43. The endoscopic surgical assembly of claim 42 wherein the at least one capture means includes a portion of a wall of the distal end of the cannula which extends into the channel to form a rim around the distal end of the cannula.

44. The endoscopic surgical assembly of claim 42 wherein the at least one capture member comprises an absorbent material connected to a portion of a wall of the distal end of the cannula.

45. The endoscopic surgical assembly of claim 28 wherein the at least one capture means of the cannula is positioned at a proximal end of the cannula.

46. An endoscopic surgical procedure, the procedure including the steps of:
(i) inserting the endoscopic surgical instrument of claim 1 into a body cavity;
(ii) sampling matter present within the body cavity; and
(iii) analysing the matter to determine if it is characteristic of damage to a structure within the body cavity.

47. The endoscopic surgical procedure of claim 46 wherein the sampling step is continuously or periodically performed throughout the endoscopic procedure.

48. The endoscopic surgical procedure of claim 46 wherein the step of analysing the matter is continuously or periodically performed during the endoscopic procedure.

49. The endoscopic surgical procedure of claim 46 wherein the gaseous contents of the body cavity are sampled and subsequently analysed.

50. The endoscopic surgical procedure of claim 46 wherein the step of sampling and/or the step of analysing matter is performed immediately after insertion of the surgical instrument into the body cavity.

51. The endoscopic surgical procedure of claim 46 wherein the step of sampling and/or the step of analysing matter is performed immediately prior to withdrawal of the surgical instrument at the end of the procedure.

52. A method for detecting damage to a structure of a body of a patient during an endoscopic procedure including the steps of:
(i) advancing the surgical assembly of claim 28 through a body cavity wall of a patient so as to create an entry port;
(ii) correctly positioning the assembly for surgical access to a target site and subsequently removing the trocar from the channel of the cannula; and
(iii) analysing the contents of the capture means of the trocar to establish whether damage has occurred to a structure of the body.

53. The method of claim 52 further including the steps of:
(iv) inserting the insufflation device of the claim 22 through the cannula and insufflating the body cavity with a suitable gas or liquid;
(v) withdrawing the insufflation device and analysing the contents of the at least one capture means of the insufflation device to establish whether damage has occurred to a structure of the body; and
(ii) withdrawing the cannula at the end of the endoscopic procedure and analysing the contents of the at least one capture means of the cannula.

54. The endoscopic surgical instrument of claim 1, the insufflation device of claim 22 and the endoscopic surgical assembly of claim 28 for use in laparoscopy, arthroscopy, thoracoscopy and hysteroscopy procedures.
